(19) United States
(12) Patent Application Publication
(10) Pub. No.: US 2023/0293190 A1
(43) Pub. Date: Sep. 21, 2023

(54) Tool for a Multiligament Reconstruction Surgery

(71) Applicant: Dr. Vandevenne Radiologie BV, Kortessem (BE)

(72) Inventors: Jan Vandevenne, Genk (BE); Kristof Smeets, Bilzen (BE); Fernand Gryspeirt, Geraardsbergen (BE)

(21) Appl. No.: 18/011,250

(22) PCT Filed: Jun. 30, 2021
(86) PCT No.: PCT/IB2021/055847

§ 371 Date: Dec. 19, 2022

(30) Foreign Application Priority Data
Jun. 30, 2020 (BE) BE2020/5487

(51) Int. Cl. A61B 17/17; A61B 17/15

(52) U.S. Cl. CPC A61B 17/1714; A61B 17/15

(57) ABSTRACT

Guide for preventing convergence between two or more drilling tunnels and/or drill pins and/or surgical instruments, which move through the drilling tunnels or are placed in them during a multi-ligament repair operation such as ACL and ALL, comprising a body (11) with at least two flat slots (21, 22, 23) extending from the proximal (P) to the distal (D) end of the body; the slots parallel to each other and separated from each other in an inferior superior direction (1-S) of the body, by distance greater than the sum of the radii of the instruments. The body at the proximal end has a profile consisting of two curved parts that protrude into the proximal direction of the body and meet in a central crossroads; and a central axis divides the body into two parts through the central intersection, the curved part of a part of the body protrudes further into the proximal direction than the curved part of the other part of the body.

### TECHNICAL FIELD

The technical field of the invention is that of surgical tools, in particular tools for use in a procedure for reconstructing multiple ligaments, for example when positioning and attaching an ACL graft and an ALL graft.

### TECHNOLOGICAL BACKGROUND OF THE INVENTION

When placing an anterior cruciate ligament (ACL) graft, it is attached on the proximal side of the femur (the upper leg bone). To attach the graft to the femur bone, a tunnel is drilled from the intercondylar area superolaterally into the distal femur diaphysis (ACL tunnel). The orientation and diameter of the ACL tunnel has to be adjusted according to the individual's anatomy.

A problem can arise when a second tunnel has to be drilled into the distal femur, in particular for positioning and attaching an anterolateral ligament (ALL). This second tunnel (ALL tunnel) is drilled into the distal femur and has an entry point through the cortical bone in the vicinity of the lateral epicondyle of the femur. It is apparent from a number of studies that convergence regularly occurs in the distal femur between the ACL tunnel and the ALL tunnel (ACL-ALL tunnel convergence), which can affect the integrity of the structures in these tunnels, such as the ACL and/or ALL graft or attachment material. This can result in all sorts of complications and even in the failure of the surgery carried out.

Current studies are primarily focused on adjusting the method of the reconstruction surgery. For example, Jaeckers et al (in Am. J. Sports. Med. 2019; 47(9):2110-2115) show that there is a significant risk of ACL-ALL tunnel convergence when using the Lemaire procedure, but that this is lower with the Macintosh procedure. Moatshe et al. (in Am. J. Sports. Med. 2017; 45(3):563-569), Perelli et al (in Arthroscopy. 2020; 36(3):776-784) and Smeets et al. (in Knee. 2019; 26(5):962-968 and in Knee Surg Sports Traumatol Arthrosc. 2019; 27(2):611-617) argue that the risk of ACL-ALL tunnel convergence can be reduced by increasing the drilling angle between the tunnels, but even this provides no guarantee.

Performing a multiligament reconstruction surgery according to the prior art thus allows the risk of ACL-ALL tunnel convergence to be reduced, but it does not tackle the root of the problem. As a result, it is often chosen to limit the depth of the tunnel for attaching the ALL, which limits the stability of the graft or attachment material and thus does not allow the ALL to be secured in a manner that is robust under tension.

A similar problem can also arise when positioning and attaching other knee ligaments, such as the lateral collateral ligament (LCL), the posterior cruciate ligament (PCL), medial collateral ligament (MCL), or similar multiligament reconstruction surgery on other parts of the body, such as in a shoulder, elbow, wrist, hip, knee, ankle, head, spinal column, thorax, abdomen, pelvis, etc. Here again there are no solutions that tackle the root of the problem.

There is therefore a need for a solution that can overcome, or at least alleviate, the drawbacks of the prior art. In particular, there is a need for a solution that prevents ACL-ALL tunnel convergence or at least reduces the risk thereof. However, this solution must be able to meet the specific conditions of the procedure, such as adjustment to the individual's unique anatomy. Preferably, the solution should also afford advantages with respect to the requirements of medical staff, such as surgeons, mainly by providing improved user-friendliness and efficiency without complicating the multiligament reconstruction surgery.

### SUMMARY OF THE INVENTION

To address one or more of the above-described needs in the field of multiligament reconstruction surgery, a surgical tool was developed by the inventors. In particular, the invention relates to a tool that can be used in a reconstruction procedure for positioning and attaching multiple ligaments. One preferred embodiment of the invention relates to a tool that is suitable for multiligament reconstruction surgery for positioning and attaching at least two grafts in a knee, such as an ACL graft, an ALL graft, an LCL graft, a PCL graft, an MCL graft, or similar multiligament reconstruction surgery on other parts of the body, such as in a shoulder, elbow, wrist, hip, knee, ankle, head, spinal column, thorax, abdomen, pelvis, etc. In addition, a method was also developed for the use of the tool in a multiligament reconstruction surgery or on training objects. In addition, a method for producing the tool was also developed.

The present invention relates to a tool and methods for preventing convergence between two or more drill tunnels and/or objects, such as surgical instruments, which move through the drill tunnels or are placed therein during a multiligament reconstruction surgery. The inventive concept of the present invention is based on the principle that a collision between two moving objects in a three-dimensional (3D) space can be avoided by determining three variables. In particular: (1) determining the orientation of the axis of linear movement of the first object in a 3D space, (2) determining the starting point or the ingress of the second object in the same 3D space, and (3) determining the maximum transverse diameters of the two moving objects. Once these variables are known, a 2D plane can be established in which free movement (for example linearly, sideways, in a curve, etc.) of the second object is possible without risk of collision with the first object.

In one aspect, the invention relates to a tool comprising a body with a proximal (P) and distal (D) end; wherein the distal end (D) of the body is configured to be placed against a surgical surface; wherein the body is provided with at least two planar slots which extend from the proximal (P) to the distal (D) end; which slots are arranged parallel to one another and are separated from one another in an inferior-superior direction (I-S) of the body; wherein the at least two planar slots are configured for the insertion of surgical instruments; and wherein the at least two planar slots are configured to set a distance in the inferior-superior direction (I-S) of the body between the surgical instruments which can be inserted into each of the at least two planar slots.

In one aspect, the invention relates to a tool comprising a body with a proximal (P) and distal (D) end; wherein the distal end (D) of the body is configured to be placed against a surgical surface; wherein the body is provided with at least two planar slots, which are arranged parallel to one another and extend from the proximal (P) to the distal (D) end of the body, and are separated from one another in an inferior-superior direction (I-S) of the body;

wherein the at least two planar slots are configured to set a distance in the inferior-superior direction (I-S) of the body between the two or more surgical instruments which can be inserted into each of the at least two planar slots.

wherein the distance in the inferior-superior direction (I-S) of the body between the at least two planar slots is greater than

In one preferred embodiment, the distance in the inferior-superior direction (I-S) of the body between at least two planar slots is greater than the sum of the radii of at least two surgical instruments, such as drill pins, which are inserted into each of the planar slots.

In one preferred embodiment, the distance in the inferior-superior direction (I-S) of the body between at least two planar slots is greater than the sum of the radii of at least two passages, such as drill tunnels, which can or will be formed or drilled by the at least two surgical instruments.

In one preferred embodiment, the distance in the inferior-superior direction (I-S) of the body is greater than the sum of the radii of two or more surgical instruments, such as drill pins, which can be inserted into each of the planar slots.

In one preferred embodiment, the distance in the inferior-superior direction (I-S) of the body is greater than the sum of the radii of two or more passages, such as drill tunnels, which can be formed, preferably drilled, by the two or more surgical instruments.

In one preferred embodiment, the distance in the inferior-superior direction (I-S) of the body between at least two planar slots is at least 1 mm to at most 100 mm; preferably 2 mm to 90 mm, or 3 mm to 80 mm, or 4 mm to 70 mm, or 5 mm to 60 mm; more preferably 5 mm to 30 mm; for example, 10 mm, 15 mm or 20 mm.

In one preferred embodiment, the body is provided with at least a third planar slot, which substantially overlaps in the inferior-superior direction with at least one planar slot to form at least two overlapping planar slots.

In one preferred embodiment, the at least three planar slots are arranged so that a central slot is formed and the at least two overlapping slots are arranged on an inferior side and on a superior side of this central slot.

In one preferred embodiment, the two overlapping slots are situated an equal inferior-superior distance away from the central planar slot.

In one preferred embodiment, the width of at least one planar slot in the inferior-superior direction (I-S) of the body is at least 1 mm to at most 50 mm; preferably 2 mm to 40 mm, or 3 mm to 30 mm, or 4 mm to 20 mm, or 5 mm to 10 mm; for example, 6 mm or 8 mm.

In one preferred embodiment, the width of each planar slot in the inferior-superior direction (I-S) of the body is at least 1 mm to at most 10 mm; preferably 2 mm to 9 mm, or 3 mm to 8 mm, or 4 mm to 7 mm; for example, 5 mm or 6 mm.

In one preferred embodiment, at least one planar slot is laterally closed so that it forms a closed planar slot on the proximal end of the body.

In one preferred embodiment, at least one planar slot is laterally closed so that it forms a closed planar slot on the distal end of the body.

In one preferred embodiment, at least one planar slot is laterally closed so that it forms a closed planar slot on the proximal end of the body and the distal end of the body.

In one preferred embodiment, the length of the closed planar slot on the distal end of the body is shorter than on the proximal end of the body.

In one preferred embodiment, the body has a profile on the proximal end consisting of two curved portions which project in the proximal direction of the body and converge at a central intersection; and wherein a central axis through the central intersection divides the body into two parts.

In one preferred embodiment, the curved portion of one part of the body projects further in the proximal direction than the curved portion of the other part of the body.

In one preferred embodiment, at least one part of the body at least partially has an inclined profile on the distal end.

In one preferred embodiment, the inclined profile on the distal end forms an angle of at least 25° to at most 75° relative to the central axis; preferably at least 35° to at most 65°; for example, 45°, 50°, 55° or 60°.

In one preferred embodiment, a proximal end of at least one planar slot is substantially limited to one part of the body.

In one preferred embodiment, the proximal ends of each planar slot are substantially limited to one part of the body so that the distal ends of each planar slot are laterally separated from one another.

In one preferred embodiment, a distal end of at least one planar slot is substantially limited to one part of the body.

In one preferred embodiment, the distal ends of each planar slot are substantially limited to one part of the body so that the proximal ends of each planar slot are laterally separated from one another.

In one preferred embodiment, a side of the body, preferably an inferior and/or superior side, is provided with markings or indicators for adjusting the orientation and/or positioning of the body and/or inserted surgical instruments.

In another aspect, the invention relates to a kit for a multiligament reconstruction surgery, comprising: the tool as described herein; at least two surgical instruments; and wherein the tool is configured for adjusting the orientation and/or the freedom of movement of the surgical instruments which are inserted into at least two planar slots of the tool.

In one preferred embodiment, the surgical instrument comprises a surgical drill or a drill pin.

In one preferred embodiment, the surgical instrument comprises a surgical pin which is provided with a graft and/or attachment material for a graft.

In another aspect, the invention relates to a method for using the kit tool as described herein, the method comprising the steps of:
(a) determining the linear axis of a first surgical instrument;
(b) positioning a distal end of the tool against a surgical surface; wherein the first surgical instrument is inserted into a first planar slot of the tool;
(c) determining the starting point or the ingress of the second surgical instrument; wherein the second surgical instrument is inserted into a second planar slot of the tool; and
wherein a distance in the inferior-superior direction (I-S) of the tool between the surgical instruments is set by the at least two planar slots.

In one preferred embodiment, the distance in the inferior-superior direction (I-S) of the tool between the surgical instruments is set in advance.

In one preferred embodiment, the distance is set in step (d) to be greater than the sum of the radii of at least two surgical instruments, such as drill pins, which have been or are inserted into each of the planar slots.

In one preferred embodiment, the distance is set in step (d) to be greater than the sum of the radii of at least two passages, such as drill tunnels, which are formed or drilled by the at least two inserted surgical instruments.

In another aspect, the invention relates to a method for producing the tool as described herein.

In one preferred embodiment, the method comprises:
at least partially producing the tool comprising at least two planar slots; and
adjusting a distance in an inferior-superior direction (I-S) of the tool between the at least two planar slots.

In one preferred embodiment, the method comprises:
(i) producing at least part of a body (10) with a proximal (P) and distal (D) end, wherein the body (10) is provided with at least two planar slots (21, 22), which are arranged parallel to one another and extend from the proximal (P) to the distal (D) end of the body (10), and are separated from one another in an inferior-superior direction (I-S) of the body (10); and
(ii) adjusting a distance in an inferior-superior direction (I-S) between the two or more planar slots (21, 22) provided in the body (10) in order to be greater than
   the sum of the radii of two or more surgical instruments, such as drill pins, which can be inserted into each of the planar slots (21, 22), and/or
   the sum of the radii of two or more passages, such as drill tunnels, which can be formed, preferably drilled, by the two or more surgical instruments;
(iii) if necessary, producing the remaining part of the tool.

In one preferred embodiment, the method comprises a step for adjusting a distance in an inferior-superior direction (I-S) of the tool, in particular the body, between at least two planar slots to at least the sum of the radii of at least two surgical instruments, such as drill pins.

In one preferred embodiment, the method comprises a step for adjusting a distance in an inferior-superior direction (I-S) of the tool, in particular the body, between at least two planar slots to at least the sum of the radii of at least two passages, such as drill tunnels, which will be formed or drilled by the at least two surgical instruments.

In one preferred embodiment, a distance in an inferior-superior direction (I-S) of the tool, in particular the body, between at least two planar slots is adjusted before producing the tool.

In one preferred embodiment, a distance in an inferior-superior direction (I-S) of the tool, in particular the body, between at least two planar slots is determined before producing the tool.

In one preferred embodiment, the tool, in particular the body, is at least partially printed by means of a 3D printer.

In one preferred embodiment, the method comprises:
providing a 3D printer;
providing a composition suitable for 3D printing; and
3D-printing a tool as described herein.

### DESCRIPTION OF THE FIGURES

In order to better indicate the features of the invention, in the attached figures some examples of possible and preferred embodiments of the present invention are described, with no limiting character. In these figures, the same numbers indicate identical or similar elements. The numerical references are discussed in more detail in the examples.

Throughout the description, claims and figures, the following numbering is retained: (10)—body; (P)—proximal end of the body; (D)—distal end of the body; (I-S) inferior-superior direction of the body; (L)—lateral direction of the body; (21)—first or central planar slot; (22)—second or superior planar slot; (23)—third or inferior planar slot.

FIG. 1 is a rear view in profile of a tool according to one preferred embodiment of the present invention.

FIG. 2 is a front view in profile of a tool according to one preferred embodiment of the present invention.

FIG. 3 is a side view in profile of a tool according to one preferred embodiment of the present invention.

FIG. 4 is a rear view of a tool according to one preferred embodiment of the present invention.

FIG. 5 is a front view of a tool according to one preferred embodiment of the present invention.

FIG. 6 is a side view of a tool according to one preferred embodiment of the present invention.

FIG. 7 is a drawing of a tool according to one preferred embodiment of the present invention with the preferred dimensions in cm.

### DETAILED DESCRIPTION

As used hereinbelow in this text, the singular forms “a”, “an” and “the” comprise both the singular and the plural, unless the context clearly denotes otherwise.

The terms “comprise”, “comprises” as used hereinbelow are synonymous with “inclusive”, “include” or “contain”, “contains” and are inclusive or open, and do not exclude additional items, elements or method steps which have not been mentioned. The terms “comprise”, “comprises” are inclusive of the term “contain”.

The enumeration of numerical values by means of ranges of figures comprises all values and fractions included in these ranges as well as the cited end points.

All documents cited in the present specification are hereby incorporated by reference in their entirety.

Unless otherwise defined, all terms disclosed in the invention, including technical and scientific terms, have the meanings which those skilled in the art usually give them. As a further guide, definitions have been incorporated in order to further explain terms which are used in the description of the invention. The terminology used herein is therefore not intended to be limiting.

In the following passages, various aspects of the invention are defined in more detail. Preferred features and embodiments of the tool according to the invention are set out hereinbelow. Each feature or embodiment of the invention that is defined as such can be combined with any other feature or embodiment unless explicitly stated otherwise. In particular, each feature that is identified as advantageous or preferred can be combined with any other feature, features or embodiments that are specified as being advantageous or preferred.

In particular, a feature indicated as “preferred” or “advantageous” may be combined with other features or properties described as “preferred” and/or “advantageous”. Reference in this specification to “one embodiment” or “an embodiment” means that a specific function, structure or characteristic described in connection with the embodiment is applicable in at least one embodiment of the present invention. Where the phrases “in one embodiment” or “an embodiment” appear at various points in the specification, they do not necessarily refer to the same embodiment although this is not excluded. Also, the features, structures or characteristics described may be combined in any suitable fashion, as will be clear to a person skilled in the art, on the basis of this description. The embodiments described and claimed in the claims may be used in any combination.

In the present description of the invention, reference is made to the appended drawings which form part thereof and which illustrate certain specific embodiments of the invention. Numerals linked to specific elements illustrate the elements concerned as examples without thereby restricting the elements. It shall be understood that structural or logical changes may be made without departing from the scope of protection of the present invention.

As described hereinabove, there is a need in the field of ligament reconstruction surgery to prevent or reduce the risk of convergence between two or more drill tunnels and/or surgical instruments, such as drill pins, which move through the drill tunnels or are positioned therein. To that end, the present invention relates to tools and methods which provide a solution to the above-described needs. Additionally, adjustments to the tool are also provided in the form of preferred embodiments, which make it possible, inter alia, to improve the efficiency, user-friendliness and usability of the tool.

One preferred embodiment of the invention relates to a tool that is suitable for a multiligament reconstruction surgery for positioning and attaching at least two grafts in a knee, such as an anterior cruciate ligament (ACL) graft, an anterolateral ligament (ALL) graft, an (ALL) graft, a lateral collateral ligament (LCL) graft, a posterior cruciate ligament (PCL) graft, and/or a medial collateral ligament (MCL) graft.

An additional advantage of the tool is that a drill tunnel can be drilled deeper in a multiligament reconstruction surgery, for example an ALL tunnel through the femur to through the contralateral cortex, which can allow the graft or attachment material to be attached more stably and thus the ligament graft can be secured in a manner that is more robust under tension. The ligament graft can, for example, be attached or secured by synthetic or biological attachment means. However, a person skilled in the art understands that the tool described herein can be adapted for multiligament reconstruction surgery for the positioning and attachment of multiple ligaments on other parts of the body, such as a shoulder, elbow, wrist, hip, knee, ankle, head, spinal column, thorax, abdomen, pelvis, etc.

The inventive concept of the present invention is based on the principle that parallel two-dimensional (2D) planes cannot come into contact with one another within a well-defined three-dimensional (3D) space. This concept can be extended to prevent a collision between two moving objects by limiting the movement of each moving object to a separate 2D plane, and by making the distance between these two parallel 2D planes greater than the sum of half of the diameters of these two moving objects measured perpendicular to each 2D plane.

In practice, this means that three variables have to be determined. These variables are (1) the orientation of the axis of linear movement of the first object in a 3D space, (2) the starting point of the movement of the second object in the same 3D space, and (3) the maximum transverse diameters of both objects. Once these variables are known, a 2D plane can be established in which free movement (for example linearly, sideways, in a curve, etc.) of the second object is possible without risk of collision with the first object.

In a first aspect, the present invention provides a tool that is suitable for preventing convergence between two or more drill tunnels and/or surgical instruments, such as drill pins, which move through these drill tunnels or are positioned therein, in a multiligament reconstruction surgery by adjusting the orientation and/or freedom of movement of these surgical instruments when they are inserted or positioned inside the tool described hereinbelow. To that end, the tool can comprise a body with a proximal (P) and distal (D) end; wherein the distal end (D) of the body is configured to be placed against a surgical surface; wherein the body is provided with at least two planar slots which extend from the proximal (P) to the distal (D) end; which slots are arranged parallel to one another and are separated from one another in an inferior-superior direction (I-S) of the body; wherein the at least two planar slots are suitable for the insertion of surgical instruments; and wherein the at least two planar slots are configured to set a distance in the inferior-superior direction (I-S) of the body between the surgical instruments which are inserted into each of the at least two planar slots.

The surgical instruments can comprise a surgical drill or a drill pin, or a surgical pin which is provided with a graft and/or attachment material for a graft. It is understood that preferred embodiments of the surgical instruments as described herein are also preferred embodiments of the tool. The tool as described herein can thus be considered a surgical tool.

The body can have a proximal and distal side, which correspond to a proximal (P) and distal (D) end, respectively. The distal side can have a distal surface that is suitable for positioning against a body surface such as a bone, joint, skin or another body part. The distal direction as used herein is the direction pointing toward this body surface. Equivalently, the proximal direction as used herein is the direction pointing away from this body surface. A surgical instrument can be inserted into the body along the proximal side so that it can exit the body from the distal side, or vice versa. The direction of the surgical instrument as it passes through the body is defined as the proximal-distal direction (P-D) or, equivalently, the distal-proximal direction. Reference is made to the associated figures for clarification of the proximal-distal direction (P-D).

The body can have an inferior and superior side, which correspond to an inferior and superior end, respectively. The directional axis of the inferior-superior direction is defined perpendicular to the proximal-distal direction. The movement of the surgical instrument will be at least partially, preferably completely, blocked in the inferior-superior direction (I-S) as the surgical instrument passes through a planar slot of the body. Reference is made to the associated figures for clarification of the inferior-superior direction (I-S).

A planar slot as used herein refers to a narrow opening in the side of the tool which extends from the proximal to the distal end of the tool. The width of the slot, i.e., the distance between the two edges of the same slot along the inferior-superior direction of the body, is suitable for the insertion of a surgical instrument. The diameter of a surgical instrument, such as a drill or drill pin, typically varies from 1 mm to 15 mm, such as 4 mm or 6 mm. A person skilled in the art understands that the width of a planar slot, the slot width, can be adjusted in a straightforward manner without negatively affecting the technical effect thereof described herein.

The planar slot ensures that the freedom of movement of an inserted instrument is limited to a two-dimensional (2D) plane that extends along the space of the slot, i.e. that it will be possible to modify the position of an inserted instrument along two perpendicular axes, in particular along the x-axis in the distal direction (in the direction of the bone) or the proximal direction (away from the bone), or sideways along the y-axis in the central direction (in the direction of the central axis of the body) or the lateral direction (away from the central axis of the body). These movements can be combined with a rotation of the instrument within this plane. However, it will not be possible to modify the position of an inserted instrument along the z-axis in the inferior-superior direction (from one planar slot toward another planar slot) or to modify the inclination of the instrument.

The width of a planar slot will preferably be adapted to the diameter of the surgical instrument, so that the instrument can move and/or rotate smoothly in the slot, but cannot be inclined. Preferably, the width of at least one planar slot, preferably of each planar slot, in the inferior-superior direction (I-S) of the body can be at least 1 mm to at most 50 mm; preferably 2 mm to 45 mm, or 2 mm to 40 mm, or 2 mm to 35 mm, or 2 mm to 30 mm, or 2 mm to 25 mm; more preferably 3 mm to 20 mm, or 4 mm to 20 mm; for example 6 mm, or 8 mm, or 10 mm, or 12 mm, or 14 mm, or 16 mm, or 18 mm.

The at least two planar slots are separated from one another in the inferior-superior direction. The distance between the two planar slots in the inferior-superior direction, the inter-slot distance, can be measured as the distance from a wall of a first slot which is facing in the direction of a second slot, for example the inferior wall, to a wall of the second slot which is facing in the direction of the first slot, for example the superior wall. The inter-slot distance therefore corresponds to the shortest distance between the at least two planar slots. The inter-slot distance can be formed by a partition which separates the two at least two planar slots in the inferior-superior direction from one another.

The inter-slot distance can be adjusted to prevent a convergence or collision between two surgical instruments or between two passages formed by these surgical instruments in the body, such as drill tunnels drilled by surgical drills. To that end, the inter-slot distance can be adapted to the diameter of the surgical instruments. In particular, the inter-slot distance can preferably be greater than the sum of the radii (i.e., half of the diameters) of at least two surgical instruments, such as drills, which are inserted into the at least two planar slots. Alternatively, the inter-slot distance can be adapted to the diameter of the passages such as drill tunnels which are formed or drilled by these surgical instruments in the bone. In particular, the inter-slot distance can preferably be greater than the sum of the radii (i.e., half of the diameters) of the passages or drill tunnels.

The inter-slot distance between at least two planar slots can be, for example, at least 1 mm to at most 100 mm; preferably 2 mm to 95 mm, or 2 mm to 90 mm, or 3 mm to 85 mm, 3 mm to 80 mm, or 4 mm to 75 mm, or 4 mm to 70 mm, or 5 mm to 65 mm, or 5 mm to 60 mm, or 5 mm to 55 mm, or 5 mm to 50 mm; more preferably 5 mm to 45 mm, or 5 mm to 40 mm, or 5 mm to 35 mm, or 5 mm to 30 mm, or 5 mm to 25 mm, or 5 mm to 20 mm; for example 8 mm, or 10 mm, or 12 mm, or 14 mm, or 16 mm, or 18 mm. A person skilled in the art understands that the inter-slot distance between at least two planar slots can be adjusted in a straightforward manner without negatively affecting the technical effect thereof described hereinbelow.

Preferably, at least one planar slot can be laterally closed, i.e., closed in the lateral direction (L) of the body, so that it forms a closed planar slot on a distal and/or proximal end of the body.

The closed slot can serve as a closure in order to partially limit the freedom of movement of an inserted surgical instrument in the lateral direction. The lateral closure is preferably present in the case of a central slot, because a surgical instrument placed in this slot will be moved mainly linearly. In addition, the closure prevents an inserted surgical instrument from being able to slide out of the closed slot when the tool is moved.

Preferably, the length of a planar slot on the distal end of the body can be shorter than on the proximal end. The planar slot can therefore take the shape of a funnel. This shape serves to partially limit the freedom of movement of an inserted surgical instrument in the lateral direction at the distal end, while still providing more extensive freedom of movement at the proximal end thereof.

The body can further be provided with at least a third planar slot; wherein the third planar slot extends from the proximal (P) to the distal (D) end; and wherein the third planar slot is arranged parallel to the first and second planar slots and is separated therefrom in an inferior-superior direction (I-S) of the body (10). Providing a third planar slot allows additional possibilities in the choice of a suitable plane in the inferior-superior direction and the usability of the tool to be improved.

The at least third planar slot can have other dimensions than the first and/or second planar slots. This can provide the possibility of using the tool for a multiligament reconstruction surgery in which at least three surgical instruments are used, for example for preventing or reducing the risk of convergence between three or more drill tunnels and/or objects which move through the drill tunnels or are positioned therein. A person skilled in the art understands that the tool can be expanded in the same way with a fourth planar slot, a fifth planar slot, etc.

The at least third planar slot can substantially overlap in the inferior-superior direction with at least one planar slot to form at least two overlapping planar slots. The body can thus be provided with at least one non-overlapping planar slot and at least two overlapping planar slots. The two planar slots overlap substantially in the inferior-superior direction, i.e., they extend over virtually the same area from the proximal end to the distal end in the body. The advantage thereof is that the user-friendliness of the tool is improved.

Preferably, the at least three planar slots, in particular the non-overlapping planar slot and the at least two overlapping planar slots, are arranged so that a central slot is formed, in which the at least two overlapping slots are arranged on an inferior side and on a superior side of this central slot. The central planar slot can preferably be situated virtually in the middle of the body of the tool. The two overlapping planar slots are preferably separated from the central planar slot by an equal inferior-superior distance. A virtually symmetrical body can thus be formed.

The preferred embodiment described above has the advantage that the tool can be used for different joints by rotating the tool. To make this advantage more clearly apparent, a reconstruction procedure for the right and left knee is described by way of example. In particular, in a procedure on a right femur, a surgical instrument is positioned in a first slot and also in a second slot which is arranged on the superior side with respect to the first slot. When this same tool is used for a procedure on a left femur, the second slot will be oriented on the wrong side of the femur, resulting in the body having to be rotated. However, this rotation will position the second slot on the inferior side of the body. Ordinarily a second tool would have to be used here, i.e., a tool adapted for the right femur and one for the left femur. By providing an embodiment with two overlapping planar slots arranged on both an inferior side and a superior side of the body, this tool can be used for both the right and the left femur simply by turning or rotating the body.

The body can have a profile on the proximal end consisting of two curved portions which converge at a central intersection. These two curved portions preferably project in the proximal direction of the body. When a central axis is drawn through the central intersection, the body can be divided into two parts. This central axis can correspond to the shortest distance in the proximal-distal direction of the body. These two parts can have the same shape and area, but will preferably differ as described further hereinbelow.

Preferably, the curved portion of one part of the body projects further in the proximal direction than the curved portion of the other part. The positioning of the tool can be facilitated thereby, which increases the user-friendliness of the tool.

Preferably, at least one part of the body at least partially has an inclined profile on the distal end. The orienting of a surgical instrument tool at the proximal end can be facilitated thereby, which increases the user-friendliness of the tool. The inclination of the profile on the distal end can preferably form an angle of at least 25° to at most 75° with respect to the central axis; preferably 30° to 70°, or 35° to 65°, or 40° to 60°, or 45 to 55°; for example, 45°, or 50°, or 55°, or 60°, etc.

Preferably, a proximal entry of at least one planar slot is substantially limited to one part of the body. The inserting of a surgical instrument at the proximal end can be facilitated thereby, which increases the user-friendliness of the tool. The proximal ends of each planar slot can also be substantially limited to one part of the body so that these proximal ends of each planar slot are laterally separated from one another.

Preferably, a distal end of at least one planar slot is substantially limited to one part of the body. The orienting of a surgical instrument tool at the distal end can be facilitated thereby, which increases the user-friendliness of the tool. The distal ends of each planar slot can also be substantially limited to one part of the body so that the distal ends of each planar slot are laterally separated from one another.

Preferably, an area of at least one planar slot is substantially limited to one part of the body. The area of a slot can, for example, be limited by substantially limiting both the proximal end and the distal end of this slot to one part of the body. The area of each planar slot can also be substantially limited to one part of the body so that the extents of each planar slot are laterally separated from one another. An area of a slot refers here to the slot area that extends from the proximal end to the distal end of the body.

The body can further be provided with markings or indicators for adjusting the orientation and/or positioning of the body and/or inserted surgical instruments. Preferably, the markings or indicators are provided on an inferior and/or superior side the body.

The markings can, for example, provide angles (in degrees) which give an indication of the angle that the inserted surgical instruments form with respect to one another. The angle can be measured as if the two planar slots were projected onto one another. Preferably, such markings are provided on the edges of the body.

The markings can, for example, be distances (in cm or mm) which give an indication of the distance between the inserted surgical instruments, or between the passages such as drill tunnels which are formed or drilled by these surgical instruments. For example, between the exit point of a first drill tunnel for the ACL and the entry point of a second drill tunnel for the ALL.

In another aspect, the present invention provides a kit for a multiligament reconstruction surgery as described herein, the kit comprising: the tool as described herein and at least two surgical instruments; and wherein the tool is configured for adjusting the orientation and/or the freedom of movement of the surgical instruments. It is understood that preferred embodiments of the tool as described herein are also preferred embodiments of the kit.

The surgical instruments are preferably inserted into at least two planar slots of the tool. The surgical instruments can comprise a surgical drill or a drill pin, or a surgical pin which is provided with a graft and/or attachment material for a graft. It is understood that preferred embodiments of the surgical instruments as described herein are also preferred embodiments of the kit. The kit as described herein can thus be considered a surgical kit.

The present invention is applicable to ligament reconstruction procedures in which the positioning and attachment of multiple ligaments on a surgical surface such as a joint, bone or other body part is required, in particular in which there is a risk of collision between two surgical instruments, such as surgical drills, or between two passages (tunnel convergence) formed by these surgical instruments in the body, such as drill tunnels drilled by surgical drills.

The tool can be used on training objects, such as a dummy or doll, or alternatively cadavers. This can allow the reconstruction procedure to be practiced so as to increase the chance of success of an intervention. The surface on which the surgical intervention is performed can be considered a surgical surface.

To that end, the method can comprise the following steps:
(a) determining a linear axis of a first surgical instrument;
(b) positioning a distal end of a tool as described herein against a surgical surface; wherein the first surgical instrument is inserted into a first planar slot (21) of the tool;
(c) determining the starting point or the ingress of the second surgical instrument; wherein the second surgical instrument is inserted into a second planar slot (22) of the tool;
wherein a distance in the inferior-superior direction (I-S) of the tool between the surgical instruments is set by the at least two planar slots.

The distance set between the at least two surgical instruments in the inferior-superior direction (I-S) of the tool can be adjusted so as to be greater than the sum of the radii of at least two surgical instruments which have been or are inserted into each of the planar slots; and/or than the sum of the radii of at least two passages which are formed or drilled by the at least two inserted surgical instruments.

The distance in the inferior-superior direction (I-S) of the tool between the at least two surgical instruments can be set in advance, if the diameters of the drill tunnels and/or the surgical instruments are known. If the diameters are not known or are changed during the reconstruction surgery, then this distance can still be adjusted by providing an adjusted tool or providing a means in order to adjust the distance in the tool.

Preferably, the tool is used in a reconstruction procedure for positioning and attaching an anterior cruciate ligament (ACL) graft and an anterolateral ligament (ALL) graft. An additional advantage of the use is that the ALL tunnels can thus be drilled deeper, for example through the femur to through the contralateral cortex, which allows the ALL graft or attachment material to be attached more stably and thus the ALL to be secured in a manner that is robust under tension.

The attachment can, for example, be by synthetic or biological attachment means. However, a person skilled in the art understands that the tool described herein can be adapted for reconstruction surgery for the positioning and attachment of multiple ligaments on other parts of the body, such as a shoulder, elbow, wrist, hip, knee, ankle, head, spinal column, thorax, abdomen and/or pelvis. The surface on which the surgical intervention is performed, for example the bone or joint, can be considered a surgical surface. A tunnel that is drilled into this surgical surface can be considered a passage.

To that end, the method can comprise the following steps:
(i) forming or drilling a first passage in a surgical surface, preferably an ACL tunnel in a femur; wherein a first surgical instrument at least partially protrudes from the surgical surface via the first passage;
(ii) positioning a distal end of a tool as described herein against the surgical surface; wherein the protruding first surgical instrument is inserted into a first planar slot of the tool;
(iii) preferably freely determining a suitable drilling point for a second passage in a surgical surface, preferably an ALL tunnel in the femur, wherein the drilling point corresponds to a distal exit of a second planar slot of the tool;
(iv) optionally, marking the position for forming or drilling the second passage in the surgical surface, preferably the ALL tunnel in the femur;
(v) forming or drilling a second passage in the surgical surface, preferably an ALL tunnel in the femur, wherein a second surgical instrument at least partially protrudes from the surgical surface via the second passage;
(vi) optionally, inserting the protruding second surgical instrument into the second planar slot of the tool;
(vii) optionally, deepening or more deeply drilling the first and/or second passage in the surgical surface;
(ix) positioning the graft and/or attachment material in the formed or drilled passages or tunnels according to a method of the prior art, preferably an ACL and/or ALL graft and/or attachment material.

In one preferred embodiment of the tool provided with three planar slots including at least two overlapping slots, the method can comprise a step for selecting a suitable slot from the at least two overlapping slots, for example a superior planar slot and an inferior planar slot. For further explanation regarding the use of the tool, reference is made to example 2, which describes an exemplary embodiment for a reconstruction procedure suitable for positioning and attaching an ACL graft and an ALL graft.

In another aspect, the present invention provides a method for producing the tool as described herein. It is understood that the preferred embodiments of the tool as described herein also form preferred embodiments for producing the tool. It is assumed here that a person skilled in the art understands how the method described herein can be adapted for the production of preferred embodiments of the tool.

To that end, the method for producing the tool as described herein can comprise the steps of: at least partially producing the tool comprising at least two planar slots; and adjusting a distance in an inferior-superior direction (I-S) of the tool between the at least two planar slots.

Preferably, the method comprises a step in which the inferior-superior distance between at least two planar slots is adjusted to the sum of the radii of at least two surgical instruments, such as drills, which can or will be inserted into the planar slots.

Preferably, the method comprises a step in which the inferior-superior distance between at least two planar slots is adjusted to the sum of the radii of at least two passages, such as drill tunnels, which will be formed or drilled by the at least two surgical instruments.

Preferably, the method comprises a step in which the width of at least one planar slot, preferably each planar slot, in the inferior-superior direction is adjusted to the diameter of a surgical instrument, such as a drill, which can or will be inserted into the planar slots.

A distance in an inferior-superior direction (I-S) of the tool between at least two planar slots can be adjusted during the production of the tool. To that end, the tool can be produced step by step in layers or sections. Preferably, the tool can be produced by stacking multiple layers consisting of different shapes and areas on top of one another. These layers are preferably inseparably and permanently bonded to one another. Each layer can be produced separately and then bonded to another layer by a fixing means. Preferably, the tool is produced as one piece in which the layers run into one another.

In one preferred embodiment of the tool provided with two planar slots, the tool can consist of five layers, which are numbered in the inferior-superior direction and have the properties as described hereinbelow:
in which layers 1 and 5 (i.e., the outermost layers, which form the superior and inferior side of the tool, respectively) have a whole structure;
in which layer 2 (i.e., the layer which forms the first planar slot) has a partially hollow structure, for example it has a whole structure on a first part of the body and has a hollow structure on a second part of the body;
in which layer 3 (i.e., the layer which determines the inter-slot distance between the first planar slot and the second planar slot) has a whole structure; and
in which layer 4 (i.e., the layer which forms the second planar slot) has a partially hollow structure, for example it has a hollow structure on a first part of the body and has a whole structure on a second part of the body.

In one preferred embodiment of the tool provided with three planar slots, the tool can consist of seven layers, which are numbered in the inferior-superior direction and have the properties as described hereinbelow:
in which layers 1 and 7 (i.e., the outermost layers, which form the superior and inferior side of the tool, respectively) have a whole structure;
in which layers 2 and 6 (i.e., the layers which form the two overlapping planar slots) have a partially hollow structure, preferably they have a whole structure on a first part of the body and have a hollow structure on a second part of the body;
in which layers 3 and 5 (i.e., the layers which determine the inter-slot distance between the two overlapping planar slots and the central slot) have a whole structure; and
in which layer 4 (i.e. the layer which forms the central slot) has a partially hollow structure, for example it has a hollow structure on a first part of the body and has a whole structure on a second part of the body, preferably it is provided with a closed opening in a first part of the body and/or it has a hollow structure in part of the first part and part of the second part of the body.

A distance in an inferior-superior direction (I-S) of the tool between at least two planar slots can be adjusted before producing the tool. To that end, the tool can be produced in a continuous production process. Preferably, the tool can be produced by forming a mold or 3D printer.

In one preferred embodiment, the tool is at least partially 3D-printed by means of a 3D printer. This method allows the tool to be produced quickly and simply on request. In addition, 3D-printer software can be provided which allows the variables of the tool to be adjusted in a user-friendly manner, in particular the width of each planar slot and the inter-slot distance between at least two slots.

To that end, the method can comprise the following steps:
providing a 3D printer;
providing a composition suitable for 3D printing; and
3D-printing a tool as described herein.

Preferably, the tool is printed in a 3D printer by means of selective laser sintering (SLS), The composition is then provided in powder form, i.e., sintering powder, which is melted layer by layer using a laser to form a solid product. A person skilled in the art understands that other 3D-printing technology is also suitable for the present invention.

Preferably, the tool is produced from polyamide (PA 12). Polyamide is a suitable material for 3D-printing a surgical tool. A person skilled in the art understands that other materials or combinations of materials are also suitable for the present invention. However, it is important that the material has sufficient rigidity to ensure the parallel arrangement of the planar slots when using the tool, i.e., that the tool cannot bend under pressure resulting in the loss of the parallel arrangement of the planar slots.

### EXAMPLES

With a view to better indicating the features of the invention, some preferred embodiments are described hereinbelow, by way of example with no limiting character, with reference to the attached figures. The embodiments illustrated in the figures concern preferred embodiments of the present invention and should in no way be interpreted as a restriction.

### Example 1

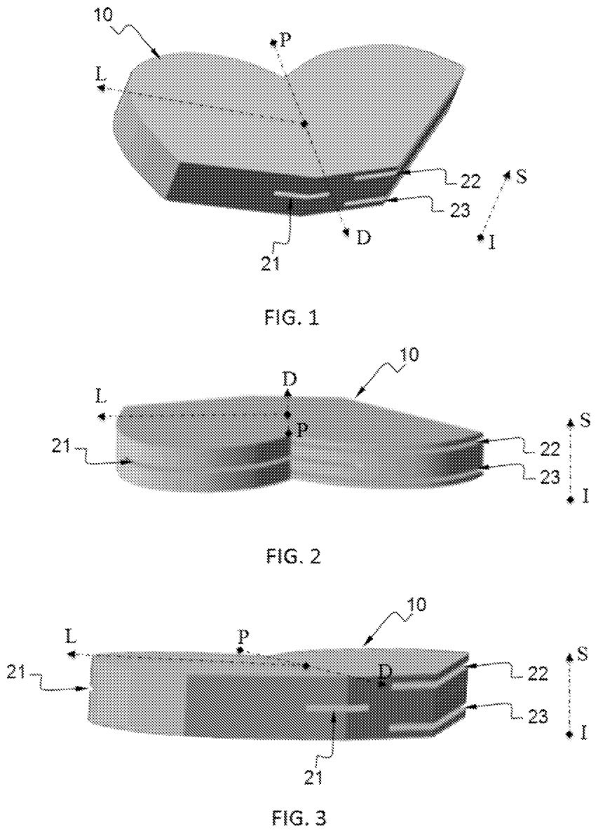

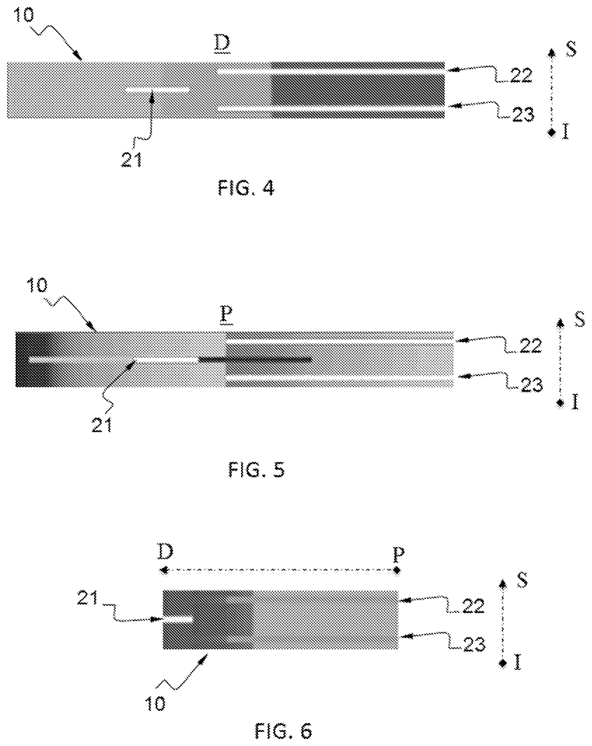

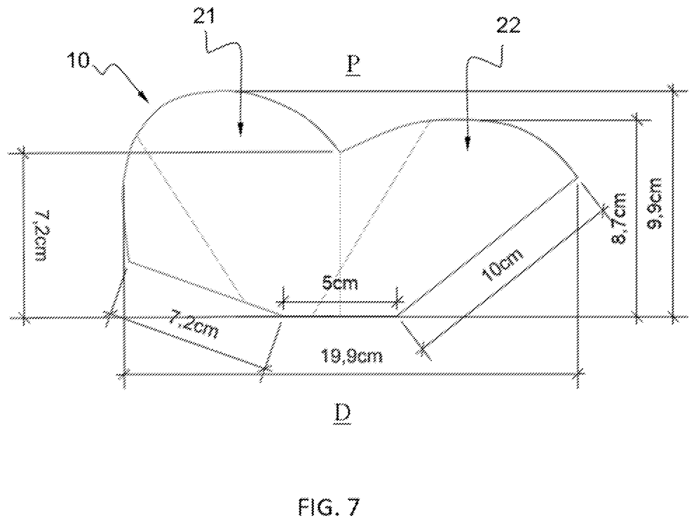

To explain the tool in more detail, reference is made to FIGS. 1 to 6. These figures show one preferred embodiment of a tool according to the present invention from different views.

The tool shown comprises here a body (10) with a proximal end (P) and a distal end (D). The body has a profile on the proximal end (P) consisting of two curved portions which project in the proximal direction of the body (10) and converge at a central intersection. A central axis through the central intersection running from the proximal end (P) to the distal end (D) divides the body (10) here into two equivalent parts.

The curved portion of the first part of the body, i.e., the left-hand part viewed from the proximal side, projects further in the proximal direction than the curved portion of the second part, i.e., the right-hand part viewed from the proximal side.

The body (10) is provided with three planar slots (21, 22, 23) which extend from the proximal to the distal end. The three planar slots (21, 22, 23) are arranged parallel to one another and are separated from one another in an inferior-superior direction (I-S) of the body (10). The axis of the inferior-superior direction (I-S) is perpendicular to the axis of the proximal-distal direction (P-D).

The first planar slot (21) is arranged in the middle of the body (10) to form a central slot (21). The second planar slot (22) and the third planar slot (23) are arranged on a superior side and on an inferior side of the body to form a superior slot (22) and an inferior slot (23), respectively. These superior (22) and inferior slots (23) substantially overlap with one another in the inferior-superior direction (I-S), i.e., they are overlapping planar slots. These superior (22) and inferior slots (23) are also separated from the central planar slot (21) by an equal inferior-superior distance.

The central slot (21) is laterally closed, with a relatively longer proximal entry/exit at the proximal end (P) of the body and a relatively shorter distal entry/exit at the distal end (D) of the body (10).

The first and the second parts partially have an inclined profile on the distal end (D). In the case of the first part, i.e., the left-hand part viewed from the distal side, the inclination is freely limited to one edge of the body, but in the case of the second part, i.e., the right-hand part viewed from the proximal side, the inclination is more pronounced.

### Example 2

To explain the use of the tool in more detail, an exemplary embodiment is described for a reconstruction procedure for positioning and attaching an ACL graft and an ALL graft in a knee. The tool used is in accordance with one preferred embodiment as described hereinabove in example 1.

The width of the planar slots (21, 22, 23) provided in the body (10) corresponds approximately to the diameter of the surgical instruments by means of which the tunnels for attaching the ACL graft, the ACL tunnel, and the ALL graft, the ALL tunnel, will be drilled. For this, a drill pin can be used with a diameter of 2.4 mm, for example.

The distance between the planar slots (21, 22, 23) corresponds approximately to the maximum transverse diameter of the final ACL and ALL tunnels. This inter-slot distance can potentially be adjusted after drilling the ACL and ALL tunnels so that the ACL and ALL grafts and/or attachment materials fit into the tunnels. The ACL tunnel can, for example, have a diameter of 8 mm and the ALL tunnel a diameter of 6 mm. The inter-slot distance is adjusted here so as to be greater than the sum of the radii of the ACL and the ALL tunnels; in the present example, this will be at least 7 mm.

The ACL tunnel is drilled using a first drill pin, the ACL drill pin, going from the intercondylar area superolaterally into the distal femur (the exit location of the ACL tunnel), and the drilling of the ACL tunnel is continued through the soft tissue of the distal upper leg until the drill pin protrudes through the skin. The body (10) is held on the lateral side of the distal upper leg and the slit-like cavity of the central slot (21) situated on distal end (D) of the body (10) is slid over the ACL drill pin. In this way, the linear axis of the ACL tunnel is determined in the body (10). The longer proximal exit of the central slot (21) allows the body (10) to be moved in the lateral direction with respect to the ACL drill pin, namely, first, a rotation with the ACL drill pin as the axis and, second, a limited translation/tilting in the plane of the central slot (21).

After this, the entry location for the tunnel for attachment of the ALL graft, the ALL tunnel, in the bone of the femur (in the vicinity of the lateral epicondyle of the femur) is freely determined by the user, such as a surgeon. Drilling can be performed at this location using a drill pin, the ALL drill pin, until just through the cortical bone of the femur. Typically, the entry location for the ALL tunnel is situated posteroinferiorly with respect to the exit location for the ACL tunnel.

The superior slot (22) or the inferior slot (23) of the body (10), which is freely chosen for optimal orientation of the body (10) or of the ALL tunnel, is then slid over the ALL drill pin. Because the superior or inferior slot (22/23) is completely open on a lateral side of the body (10), this ALL drill pin can be slid laterally into the chosen planar slot (22, 23). This lateral insertion is facilitated by virtue of the body (10) being able to be rotated and tilted around the ACL drill pin, which is fixed within the ACL tunnel.

The tool thus makes it possible to be able to drill the ALL drill pin in an anterior plane (in the case of choosing the superior slot (22)) or posterior plane (in the case of choosing the inferior slot (22)) with respect to the plane that comprises the ACL drill pin. This is an advantage that can be used when there is a risk of undesired penetration of the cortical bone on another side of the femur when drilling the ALL tunnel, such an example of undesired penetration: intra-articular in the knee joint.

The ALL drill pin is then drilled deeper into the bone of the femur following the plane dictated by the chosen superior or inferior slot (22, 23) of the body (10). The two drill pins are consequently positioned in two parallel planes; in particular, the ACL drill pin is in the central slot (21) and the ALL drill pin is in the superior or inferior slot (22, 23) which makes a collision between both drill pins impossible and consequently prevents ACL-ALL tunnel convergence.

1. A tool for preventing convergence between two or more drill tunnels and/or surgical instruments during a multiligament reconstruction surgery;
the tool comprising a body (10) with a proximal (P) and a distal (D) end; wherein the distal end (D) of the body (10) is configured to be placed against a surgical surface;
wherein the body (10) is provided with at least two planar slots (21, 22), which are arranged parallel to one another and extend from the proximal (P) to the distal (D) end of the body (10), and are separated from one another in an inferior-superior direction (I-S) of the body (10);
wherein the at least two planar slots (21, 22) are configured to set a distance in the inferior-superior direction (I-S) of the body (10) between the two or more surgical instruments which can be inserted into each of the at least two planar slots (21, 22);
wherein the distance in the inferior-superior direction (I-S) of the body (10) between the at least two planar slots (21, 22) is greater than
the sum of the radii of two or more surgical instruments, such as drill pins, which can be inserted into each of the planar slots (21, 22), and/or
the sum of the radii of two or more passages, such as drill tunnels, which can be formed, preferably drilled, by the two or more surgical instruments.

2. The tool as claimed in preceding claim 1, wherein the distance in the inferior-superior direction (I-S) between the at least two planar slots (21, 22) is at least 1 mm to at most 100 mm; preferably 2 mm to 90 mm, or 3 mm to 80 mm, or 4 mm to 70 mm, or 5 mm to 60 mm; more preferably 5 mm to 30 mm; for example, 10 mm, 15 mm or 20 mm.

3. The tool as claimed in one of the preceding claims, wherein the body (10) is provided with at least a third planar slot (23), which substantially overlaps in the inferior-superior direction (I-S) of the body (10) with at least one planar slot (22) to form at least two overlapping planar slots (22, 23).

4. The tool as claimed in claim 3, wherein the at least three planar slots (21, 22, 23) are arranged so that a central slot (21) is formed and the at least two overlapping slots (22, 23) are arranged on an inferior side and on a superior side of this central slot (21).

5. The tool as claimed in claim 4, wherein the two overlapping slots (22, 23) are situated an equal distance away from the central planar slot (21) in the inferior-superior direction (I-S) of the body (10).

6. The tool as claimed in one of the preceding claims, wherein the width of at least one planar slot, preferably of each planar slot (21, 22, 23), in the inferior-superior direction (I-S) of the body (10) is at least 1 mm to at most 50 mm; preferably 2 mm to 40 mm, or 3 mm to 30 mm, or 4 mm to 20 mm, or 5 mm to 10 mm; for example 6 mm or 8 mm.

7. The tool as claimed in one of the preceding claims, wherein at least one planar slot is laterally closed so that it forms a closed planar slot on the proximal end (P) of the body (10) and/or on the distal end (D) of the body (10).

8. The tool as claimed in claim 7, wherein the length of the closed planar slot on the distal end (D) of the body (10) is shorter than on the proximal end (P) of the body (10).

9. The tool as claimed in one of the preceding claims, wherein the body (10) has a profile on the proximal end (P) consisting of two curved portions which project in the proximal direction of the body (10) and converge at a central intersection; and wherein a central axis through the central intersection divides the body (10) into two parts.

10. The tool as claimed in claim 9, wherein the curved portion of one part of the body (10) projects further in the proximal direction of the body (10) than the curved portion of the other part of the body (10).

11. The tool as claimed in either one of claim 9 or 10, wherein at least one part of the body (10) at least partially has an inclined profile on the distal end (D); preferably wherein this inclined profile forms an angle of at least 25° to at most 75° relative to the central axis; preferably at least 35° to at most 65°.

12. The tool as claimed in one of claims 9 to 11, wherein a proximal end of at least one planar slot is substantially limited to one part of the body (10); preferably wherein the proximal ends of each planar slot are substantially limited to one part of the body (10) so that the distal ends of each planar slot are laterally separated from one another.

13. The tool as claimed in one of claims 9 to 12, wherein a distal end of at least one planar slot is substantially limited to one part of the body (10); preferably wherein the distal ends of each planar slot are substantially limited to one part of the body (10) so that the proximal ends of each planar slot are laterally separated from one another.

14. The tool as claimed in one of the preceding claims, wherein an inferior and/or superior side of the body (10) is provided with markings or indicators for adjusting the orientation and/or positioning of the body (10) and/or inserted surgical instruments.

15. A kit for a multiligament reconstruction surgery, comprising:
a tool as claimed in one of the preceding claims;
at least two surgical instruments; and,
wherein the tool is configured for adjusting the orientation and/or the freedom of movement of the surgical instruments which can be inserted into two or more planar slots (21, 22) of the tool.

16. The kit as claimed in claim 15, wherein a surgical instrument comprises a surgical drill and/or a drill pin; and/or comprises a surgical pin which is provided with a graft and/or attachment material for a graft.

17. A method for using the tool on a training object, such as a dummy, doll or cadaver, as claimed in one of the preceding claims and/or the kit as claimed in one of the preceding claims, comprising the steps of:
(a) determining a linear axis of a first surgical instrument;
(b) positioning a distal end of the tool against a surgical surface; wherein the first surgical instrument is inserted into a first planar slot (21) of the tool;
(c) determining the starting point or the ingress of the second surgical instrument;
wherein the second surgical instrument is inserted into a second planar slot (22) of the tool; and
wherein a distance in the inferior-superior direction (I-S) of the tool between the surgical instruments is set by the at least two planar slots (21, 22).

18. A method for using the tool as claimed in one of the preceding claims and/or the kit as claimed in one of the preceding claims, comprising the steps of:
(a) determining a linear axis of a first surgical instrument;
(b) positioning a distal end of the tool against a surgical surface; wherein the first surgical instrument is inserted into a first planar slot (21) of the tool;
(c) determining the starting point or the ingress of the second surgical instrument;
wherein the second surgical instrument is inserted into a second planar slot (22) of the tool; and
wherein a distance in the inferior-superior direction (I-S) of the tool between the surgical instruments is set by the at least two planar slots (21, 22).

19. The method as claimed in either one of claim 17 or 18, wherein the distance in the inferior-superior direction (I-S) of the tool between the surgical instruments is set in advance.

20. The method as claimed in one of claims 17 to 19, wherein the distance in the inferior-superior direction (I-S) of the tool between the surgical instruments is set so as to be greater than
the sum of the radii of the at least two surgical instruments, such as drill pins, which can be inserted into each of the planar slots (21, 22); and/or
the sum of the radii of at least two passages, such as drill tunnels, which can be formed, preferably drilled, by the at least two inserted surgical instruments.

21. A method for producing a tool as claimed in one of the preceding claims, comprising the steps of:
(i) producing at least part of a body (10) with a proximal (P) and distal (D) end, wherein the body (10) is provided with at least two planar slots (21, 22), which are arranged parallel to one another and extend from the proximal (P) to the distal end (D) of the body (10), and are separated from one another in an inferior-superior direction (I-S) of the body (10); and
(ii) adjusting a distance in an inferior-superior direction (I-S) between the two or more planar slots (21, 22) provided in the body (10) in order to be greater than
the sum of the radii of two or more surgical instruments, such as drill pins, which can be inserted into each of the planar slots (21, 22), and/or
the sum of the radii of two or more passages, such as drill tunnels, which can be formed, preferably drilled, by the two or more surgical instruments;
(iii) if necessary, producing the remaining part of the tool;
wherein the at least two planar slots (21, 22) are configured to set a distance in the inferior-superior direction (I-S) of the body (10) between the two or more surgical instruments which can be inserted into each of the at least two planar slots (21, 22).

22. The method as claimed in claim 21, wherein the distance in the inferior-superior direction (I-S) of the body (10) between at least two planar slots (21, 22) is determined before producing the tool.

23. The method either one of claim 21 or 22, wherein the body (10) is at least partially printed by means of a 3D printer.
